(19) United States
(12) Patent Application Publication
(10) Pub. No.: US 2026/0258335 A1
(43) Pub. Date: Sep. 3, 2026

(54) Microfluidic Device For Cell Culture And Method Of Fabricating The Same

(71) Applicant: National University of Singapore

(72) Inventors: Chwee Teck LIM; Nishanth VENUGOPAL MENON; Jee Yeon LEE

(21) Appl. No.: 18/993,338

(22) PCT Filed: Jul. 11, 2023
(86) PCT No.: PCT/SG2023/050486

§ 371 Date: Jan. 10, 2025

(30) Foreign Application Priority Data
Jul. 12, 2022 (SG) 10202250443K

(51) Int. Cl. C12M 3/06; C12M 1/00; C12M 1/34; C12M 1/42; C12M 3/00

(52) U.S. Cl. CPC C12M 23/16; C12M 21/08; C12M 23/22; C12M 23/34; C12M 29/00; C12M 35/04; C12M 41/00; C12M 41/34

(57) ABSTRACT

Microfluidic devices and method of fabricating the microfluidic devices are provided. The microfluidic device includes a base layer, a first layer disposed on the base layer and a second layer disposed on the first layer. The first layer defines one or more cell culture chambers. Each of the one or more cell culture chambers configured to contain one or more cells. The second layer defines one or more air chambers. Each of the one or more air chambers is adjacent a respective cell culture chamber. The microfluidic device allows the culture of single or multiple human-derived or animal-derived cells, or their co-culture with microorganisms under static or dynamic culture conditions.

### FIELD OF THE INVENTION

The invention generally relates to cell culture device, more particularly, microfluidic cell culture device and method of fabricating the device.

### BACKGROUND

Organ-on-chips or microphysiological systems are a class of microfluidic platforms that cater towards recreating physiologically relevant microenvironment of human or animal organs, which could be used as in vitro models for disease studies and drug development applications. The chip contains microchannels for guiding and manipulating minute volumes of solution and can often serve as effective mimics of human physiology and disease. The chips enable experimentation with biological cells and tissues outside the body. The organ-on-chips allow a higher level of control over the microenvironment that ensures tissue life support, as well as a means to directly observe cell and tissue behaviour. Existing designs of microfluidic organ-on-chips include: i) synthetic membrane-based devices; ii) ECM patterned or hydrogel-based platforms; and iii) modular or directly linked microfluidic systems.

### i) Synthetic Membrane-based Devices

A common approach for developing organ chips is the miniaturization of the traditional transwell assay. The basic design includes multiple layers of microchambers/fluidic channels that are juxtaposed and bonded one over the other while remaining separated through a porous membrane. This configuration has been widely used for the generation of different organ-on-chips including human gut and lungs. While unique shear stress can be applied to the top and bottom chambers (on either side of the porous membrane), any other actuation forces cannot be disjointed and has to be applied to both the chambers. Hence the flexibility to culture two different cells/tissues that require different physiological biomechanics, such as a liver or kidney, cannot be incorporated into the same device. Instead, multiple platforms will be required to achieve a more complex biomimetic organ setup.

Multi-layered devices have also been used to recreate other biomimetic cell culture platforms especially blood vessels and human heart. However, these platforms suffer from similar drawbacks as discussed above because only a single cell or tissue type can be maintained in one platform as the mechanical forces applied are constant across the entire platform. Another significant drawback in such membrane-based systems are the challenges with high resolution microscopy as position of the cells or tissues far greater than the working distances of conventional high magnification objective lenses.

### ii) ECM Patterned or Hydrogel-based Platforms

Extracellular matrix (ECM) patterning is an alternative approach that can be used for compartmentalizing micro-chambers. This could be done by either patterning ECM in defined channels within a polymer-based device or by creating the whole platform using ECM as the scaffolding (hydrogel-based platforms). Several organ chips including gut, liver, heart, blood vessels as well as brain have been recreated using this approach. These chips alleviate the issue of high magnification imaging as they are often single-layered devices and also the lateral arrangement of channels allows the flexibility of incorporating multiple tissue chambers side by side laterally. However, the current strategies employed for ECM patterned devices do not allow for long-term (>24 hours) continuous perfusion of fluids through these chambers using an active pumping source such as a syringe pump. This is due to the detrimental effects of active pumping on the integrity of the ECM hydrogel. Additionally, hydrogel-based platforms have tedious and time-consuming fabrication approaches and challenges to fluid handling due to the porous nature of these scaffolds. Also, the current platforms do not favor the integration of any mechanical actuation except for hydrostatic pressure-based fluid flow and hence shear stress for long-term cultures.

iii) Modular or Directly Linked Microfluidic Systems

Another methodology for the generation of organ chips is that of modular microfluidic systems whereby distinct organ systems are generated in isolation and these different organ modules are then interconnected by external tubings or using microchannels or capillaries. While modular or compartmentalized microfluidic platforms do not have the issues faced by the above discussed design considerations especially in terms of imaging or the presence of ECM hydrogel, the interconnections of the different organ chip channels using convective fluidic channels calls for complications in the maintenance of different cell types. This stems from the unique biochemical requirements of the different organ systems.

Accordingly, there is a need for an improved device which may allow greater design flexibility for easy modification and incorporation of multiple cell culture chambers for high throughput organ-on-chip applications, and also have the ability to maintain distinct mechanical stimuli and/or biochemical environments in different cell culture chambers simultaneously.

### SUMMARY OF THE INVENTION

In accordance with a first aspect of the present disclosure, a microfluidic device is provided. The microfluidic device comprises: a base layer; a first layer disposed on the base layer, the first layer defining one or more cell culture chambers, each of the one or more cell culture chambers configured to contain one or more cell cultures; and a second layer disposed on the first layer, the second layer defining one or more air chambers. Each of the one or more air chambers is adjacent a respective cell culture chamber.

In certain embodiments, the base layer comprises a transparent glass layer.

In certain embodiments, the first layer defines a plurality of cell culture chambers. The plurality of cell culture chambers is disposed in a lateral configuration.

In certain embodiments, the second layer defines a corresponding plurality of air chambers. The device further comprises a programmable pneumatic controller for operating the plurality of air chambers. The programmable pneumatic controller is configured to actuate the plurality of air chamber chambers independently of one another.

In certain embodiments, each of the plurality of air chambers is configured to generate a biomechanical cue in the respective cell culture chamber. The biomechanical cue comprises at least one of a fluidic flow, a shear stress, a pressure gradient, or mechanical compression and expansion.

In certain embodiments, at least selected ones of the plurality of cell culture chambers are connected to one another.

In certain embodiments, the first layer further defines a plurality of extracellular matrix (ECM) channels. Each of the ECM channels adjoins a respective one of the plurality of cell culture chambers.

In certain embodiments, the microfluidic device further comprises a capillary burst valve disposed between each of the plurality of ECM channels and a respective one of the plurality of cell culture members.

In accordance with a second aspect of the present disclosure, a cell culture method is provided. The method comprises: providing a microfluidic device as described above; providing one or more cell cultures in the one or more cell culture chambers; and actuating the one or more air chambers such that each of the one or more air chambers generates a mechanical stimulus on a respective cell culture chamber.

In certain embodiments, providing one or more cell cultures in the one or more cell culture chambers comprises providing a cell culture of a first cell type in a first cell culture chamber and providing a cell culture of a second cell type different from the first cell type in a second cell culture chamber.

In certain embodiments, providing one or more cell cultures in the one or more cell culture chambers further comprises providing one or more types of microorganisms together with a cell culture in a cell culture chamber.

In certain embodiments, providing one or more cell cultures in the one or more cell culture chambers further comprises controlling fluid flow in the one or more cell cultures via respective inlets and outlets of the one or more cell culture chambers.

In certain embodiments, actuating the one or more air chambers comprises actuating the one or more air chamber chambers independently of one another.

In certain embodiments, the method further comprises generating a mechanical stimulus in one or more cell culture chambers with one or more cell culture by actuating the one or more air chambers and controlling fluid flow through the one or more cell culture chambers, simultaneously.

In certain embodiments, the method further comprises controlling one or more biochemical cues selected from a group consisting of proteins, peptides, oligonucleotides, chemicals, nanoparticles or drugs, for the one or more cell cultures.

In certain embodiments, controlling one or more biochemical cues comprises tuning the oxygen concentration in the one or more cell culture chambers using an oxygen control module.

In accordance with a third aspect of the present disclosure, a method of fabricating a microfluidic device is provided. The method comprises: providing a base layer; forming a first layer such that the first layer defines one or more cell culture chambers, each of the one or more cell culture chambers configured to contain one or more cell cultures; forming a second layer such that the second layer defines one or more air chambers; disposing the first layer on the base layer; and disposing the second layer on the first layer such that each of the one or more air chambers is adjacent a respective cell culture chamber.

In certain embodiments, forming the first layer comprises spin-coating polydimethylsiloxane (PDMS) over a first mold, and wherein forming the second layer comprises casting PDMS over a second mold.

In certain embodiments, disposing the first layer on the base layer comprises a first plasma bonding step, and wherein disposing the second layer on the first layer comprises a second plasma bonding step.

In certain embodiments, forming the first layer further comprises forming a plurality of extracellular matrix (ECM) channels each adjoining a respective one of the plurality of cell culture chambers.

The microfluidic device according to the present disclosure can be used to build a platform consisting of a microscopy-grade glass coverslip and multiple layers of chips with an in-built mechanical actuator. The platform allows the culture of single or multiple human-derived or animal-derived cells or their co-culture with microorganisms under static or dynamic culture conditions of fluid flow, shear stress and cyclic-mechanical compression and relaxation. The microfluidic device provides the flexibility to tune the application and recapitulate multiple microphysiological systems such as liver, gut, blood vessel, kidney and heart. Multiple cell culture chambers with independently controllable biomechanical and biochemical cues facilitate drug or compound screening process. Further, dimensions or design of the different cell culture chambers as well as the mechanical actuator can be modified comfortably to suit different applications.

### BRIEF DESCRIPTION OF THE DRAWINGS

A full and enabling disclosure of the present invention, including various embodiments and using the present devices and methods, directed to one of ordinary skill in the art, is set forth in the specification, which makes reference to the appended figures, in which:

FIG. 1 shows a microfluidic device according to one embodiment of the present disclosure.

FIG. 2 shows a sectional view of a microfluidic device according to one embodiment of the present disclosure.

FIG. 3 shows schematic views of the air chamber and cell culture chamber in different states.

FIG. 4 depicts a graph showing various states of the cell culture chamber in response to different extents of pneumatic actuation of the air chamber.

FIGS. 5A-5B show various designs of the second layer in the microfluidic device.

FIGS. 6A-6B show various designs of the first layer in the microfluidic device.

FIG. 7 illustrates microscopic images of a microfluidic device and a cell culture chamber according to one embodiment of the present disclosure.

FIGS. 8A-8B show a microfluidic device according to another embodiment of the present disclosure.

FIGS. 9A-9B show a microfluidic device according to a further embodiment of the present disclosure.

FIG. 9C illustrates microscopic images showing the diffusion of molecules through the ECM.

FIG. 9D depicts a graph showing the fluorescence intensity over distance along an end section of the ECM channel.

FIG. 10 illustrates microscopic images showing a tri-culture system created by a microfluidic device according to one embodiment of the present disclosure.

FIG. 11 illustrates microscopic images showing bacterial colonization on a microfluidic device according to one embodiment of the present disclosure.

FIG. 12 depicts an example method of fabricating a microfluidic device according to one embodiment of the present disclosure.

FIG. 13 shows a schematic view of the fabrication process.

FIG. 14 depicts an example cell culture method according to one embodiment of the present disclosure.

### DETAILED DESCRIPTION

Reference now will be made in detail to embodiments of the present invention, one or more examples of which are illustrated in the drawings. Each example is provided by way of explanation, rather than limitation of, the invention. Features illustrated or described as part of one embodiment can be used with another embodiment to yield a still further embodiment. It is intended that the present disclosure covers such modifications and variations within the scope of the appended claims and their equivalents. The detailed description uses numerical and letter designations to refer to features in the drawings. Like or similar designations in the drawings and description have been used to refer to like or similar parts of the invention.

As used herein, the terms “first”, “second”, and “third” may be used interchangeably to distinguish one component from another and are not intended to signify location or importance of the individual components. The singular forms “a,” “an,” and “the” include plural references unless the context clearly dictates otherwise. The terms “coupled,” “fixed,” “attached to,” and the like refer to both direct coupling, fixing, or attaching, as well as indirect coupling, fixing, or attaching through one or more intermediate components or features, unless otherwise specified herein. As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus.

Terms of approximation, such as “about,” “generally,” “approximately,” or “substantially,” include values within ten percent greater or less than the stated value.

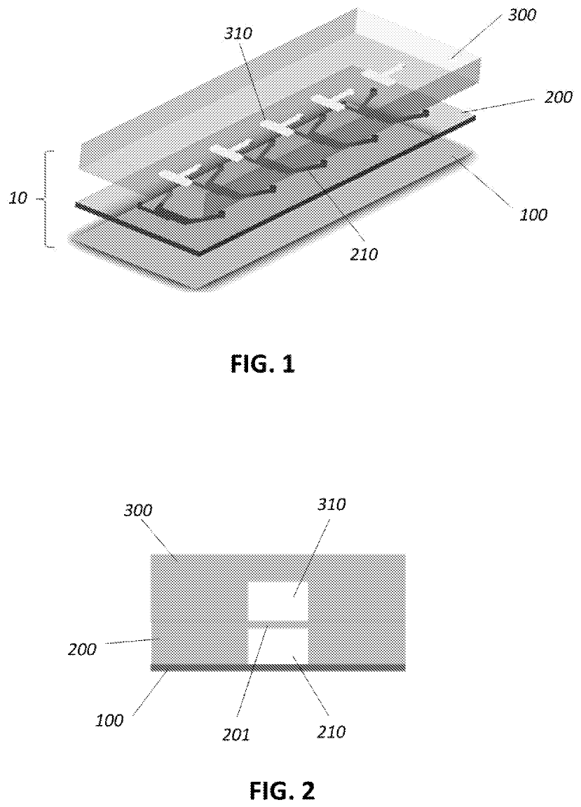

FIG. 1 shows a microfluidic device 10 according to one embodiment of the present disclosure. The microfluidic device can be used as an organ chip and includes multiple layers with different functionalities. The microfluidic device 10 in FIG. 1 has a base layer 100, a first layer disposed on the base layer 200 and a second layer 300 disposed on the first layer 200.

The base layer 100 is configured to provide a support for the other layers. The base layer 100 can be made of glass or other transparent materials. The transparent base layer enables optical analysis or observation of the cells or tissues being cultured in the first layer 200 from a bottom side of the device 10. In certain embodiments, the base layer 100 can be in the form of a glass coverslip, which makes the microfluidic device 10 easily integrable with high-resolution microscopy for real-time monitoring and optical assessment of the cell or tissue culture process. The glass coverslip may have a thickness of 80-300 μm, for example 130-170 μm, 160-190 μm, 190-250 μm. In one embodiment, the glass coverslip may have a thickness of around 150 μm, making the microfluidic device suitable for super-resolution and high magnification (100× objective) imaging without the need for electron microscopic techniques for analysis.

The first layer 200 is configured to provide space for cell culture. The first layer 200 defines one or more cell culture chambers 210. Each chamber 210 can be configured to house cells, miniature tissues, and/or microorganisms grown and residing in the microfluidic device. In the embodiment shown in FIG. 1, the first layer 200 defines an array of five cell culture chambers 210 having the same design. The cell culture chambers 210 can be arranged generally in a lateral configuration. Particularly, the cell culture chambers 210 can be equally spaced along the longitudinal direction of the microfluidic device 10. In certain embodiments, the cell culture chambers 210 are isolated from each other such that different mechanical actuation patterns can be applied to different cell culture chambers. In alternative variations, every cell culture chamber may have a unique design. It should be appreciated that number of cell culture chambers and the dimensions or shapes of the cell culture chambers can be selected to suit the cells to be cultured.

The second layer 300 functions as an actuator layer to generate and regulate a multitude of mechanical forces on the cells within the cell culture chambers in the first layer. The second layer 300 defines one or more air chambers 310. Actuation of the air chambers 310 enables the microfluidic device 10 to mimic dynamic physiological microenvironment, including, for example, heartbeat, air breathing, intestinal peristaltic movement, arteriolar constriction, etc. Similar to the first layer 200, the second layer 300 shown in FIG. 1 defines an array of five air chambers 310 having the same design. The air chamber 310 can be equally spaced along the longitudinal direction of the microfluidic device. In certain embodiments, the air chambers 310 are isolated from each other such that actuation of air chambers can be controlled independently. In alternative variations, every air chamber may have a unique design. It should be appreciated that number of air chambers and the dimensions or shapes of the air chambers can be selected according to requirements of specific applications.

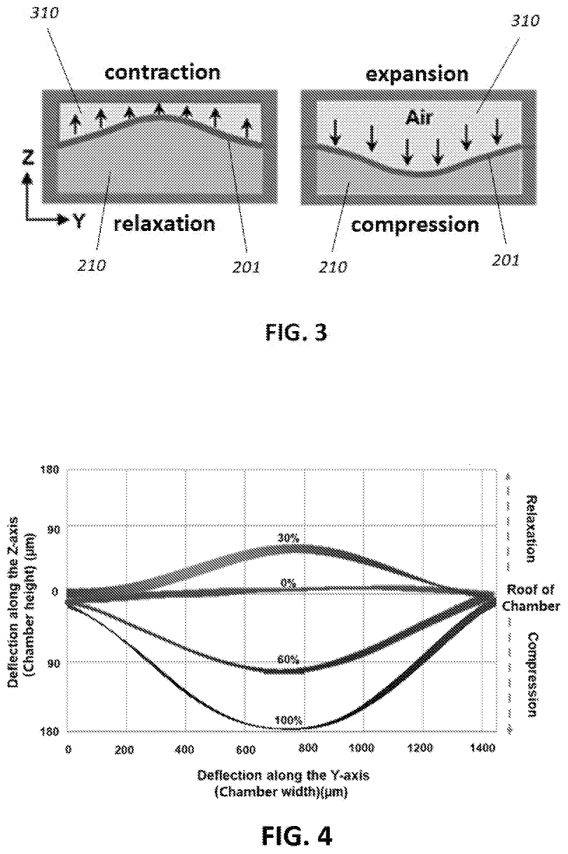

FIG. 2 shows a sectional view of a microfluidic device according to one embodiment of the present disclosure. The first layer 200 is supported on the base layer 100. The second layer 300 is disposed on the first layer 200. One cell culture chamber 210 and one air chamber 310 are visible in FIG. 2. In this embodiment, the cell culture chamber 210 is defined by the base layer 100 and the first layer 200. The cell culture chamber 210 can include a recessed cavity or microwell formed in the first layer 200 and closed by the base layer 100. The base layer 100 prevents the cells or tissues in the cell culture chamber from being exposed to atmosphere. The air chamber 310 is defined by the first layer 200 and the second layer 300. The air chamber 310 can include a microwell formed in the second layer 300 and closed by the first layer 200.

The microfluidic device 10 in FIG. 1 includes five cell culture chambers 210 and five air chambers 310. Each of the multiple air chambers 310 in the second layer 300 is positioned adjacent a respective cell culture chamber 210 such that movement of the air chamber 310 in the second layer 300 changes the microenvironment within the respective cell culture chamber 210 in the first layer 200. As shown in FIG. 2, the cell culture chamber 210 is aligned with the air chamber 310 in the vertical direction. A portion of the upper part of the first layer 200 forming the roof 201 of the cell culture chamber 210 separates the cell culture chamber 210 from the air chamber 310. It should be appreciated the two chambers can be separated by other designs of partition member.

The first and second layers 200, 300 formed with one or more chambers 210, 300 may be fabricated using techniques such as injection molding, compression injection molding, embossing, lithographic etching, laser etching, and/or other methods. Each of the first and second layers 200, 300 in the microfluidic device 10 shown in FIG. 1 is a unitary body. In alternative variations, the first and second layers can have discrete portions that are coupled together.

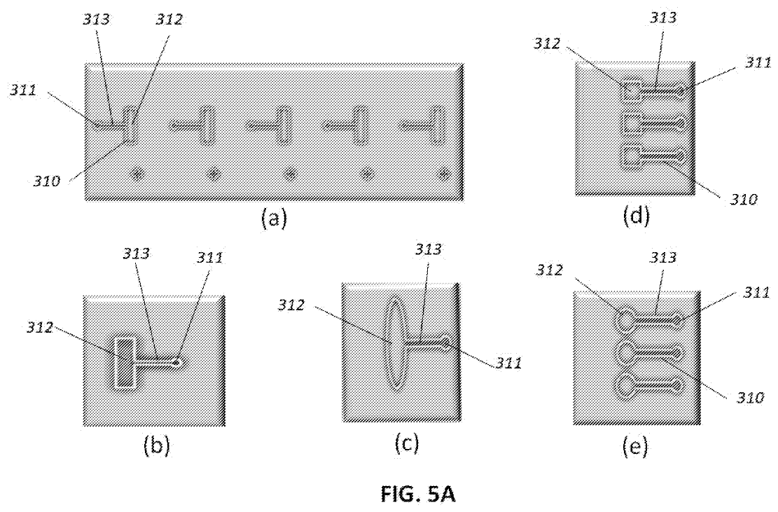

The air chamber 310 can be made to expand or contract using pneumatic actuation. As shown in FIG. 3, the percentage expansion and contraction can be controlled using a programmable pneumatic controller. The air chamber 310 acts as an actuator to generate a multitude of mechanical forces on the cells or tissues within the cell culture chamber. The mechanical forces can be controlled to cause cyclic relaxation and compression of the cell culture chamber, and/or simulate peristaltic fluid dynamics. Accordingly, the air chambers 310 generate different mechanical stimuli in the cell culture chambers 210. On the other hand, the inlets and outlets of the cell culture chambers 210 allow the perfusion of cell culture media or suitable agents such as drugs to be introduced to the cell culture chambers 210 under constant perfusion controlled by fluid handling pumps such as a syringe pump, pressure pump or peristaltic pump. Perfusion culture generates shear stress which is an essential biomechanical cue for cell growth.

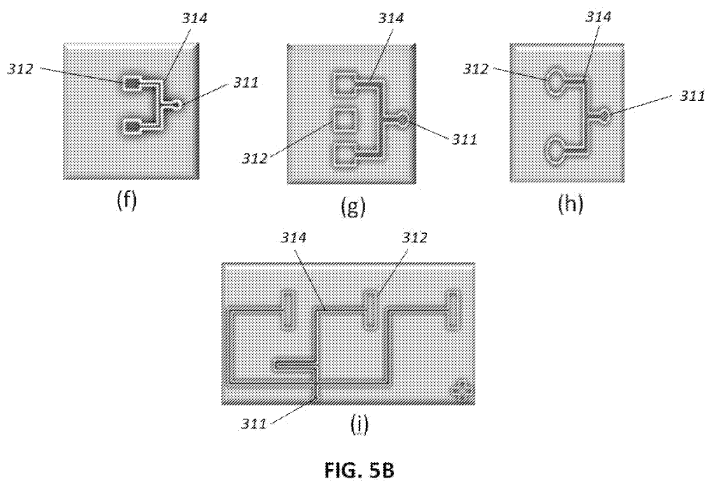

FIG. 4 depicts a graph showing various states of the cell culture chamber in response to different extents of pneumatic actuation of the air chamber. The four curves in the graph are 3D reconstructions of the microscopic images of the roof of cell culture chamber in different states in a relaxation and compression cycle. The 0% curve is generally linear and corresponds to a state in which the cell culture chamber does not experience a relaxation or compression. The 30% curve is a convex curve corresponding to a state in which the cell culture chamber is experiencing a moderate relaxation in response to a contraction of the air chamber. The 60% curve is a concave curve corresponding to a state in which the cell culture chamber is experiencing a moderate compression in response to a medium degree of expansion of the air chamber. The 100% curve is a concave curve corresponding to a state in which the cell culture chamber is experiencing a full compression in response to a high degree of expansion of the air chamber.

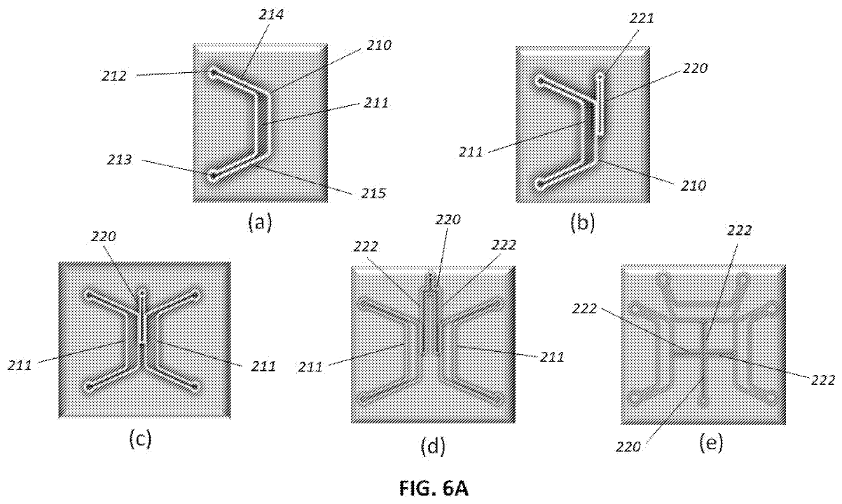

FIGS. 5A-5B illustrate various designs of the second layer in the microfluidic device. The second layer functions as actuator layer and may include one or more air chambers. The actuator layer shown in view (a) includes five air chambers 310 arranged along the longitudinal direction of the actuator layer. Each air chamber 310 has one air inlet 311, one air cavity 312 and one air passageway 313 connecting the air inlet 311 and the air cavity 312. The air passageway 313 extends in a direction perpendicular to the air cavity 312. The width of the air passageway 313 can be made smaller than the diameter of the air inlet 311 to restrict the air flow rate.

Each air chamber can be configured to generate a biomechanical cue in a respective cell culture chamber. The biomechanical cue may include at least one of a fluidic flow, a shear stress, a pressure gradient, or mechanical compression and expansion. For example, air can be introduced from the air inlet 311 to the air cavity 312 to expand the air chamber 310, which in turns compresses the cell culture chamber 210 beneath. Removing air from the air cavity causes the air chamber to contract and thereby relaxing the cell culture chamber.

Each of the actuator layers shown in views (b) and (c) has a single air chamber. The air inlet 311 can be arranged on one side of the air cavity 312. The air cavity 312 may have an elongated shape, e.g. rectangular or elliptical. A rectangular air cavity may have an aspect ratio greater than 1.5, e.g. in the range of 2 to 5. The air inlet 311 may have a round cross section. In alternative variations, each air chamber may have multiple air inlets and multiple air passageways. The one or multiple air inlets can be controlled with a programmable pneumatic controller. In certain embodiments, the pneumatic controller is configured or programmed to actuate multiple air chambers independently of one another.

Each of the actuator layers shown in views (d) and (e) has an array of three air chambers 310. The air chambers 310 can be arranged in a more compact manner compared to the design in view (a). The air cavity 312 may have a square or round shape.

Each of the actuator layers shown in views (f) to (i) includes a single air inlet 311 and multiple air cavities 312. In views (f), (h) and (i), the single air inlet 311 is connected to each air cavity 312 via a network of air channels 314. In view (g), no direct fluid communication is provided between the middle air cavity 312 and the air inlet 311. It should be appreciated that the air channel network (e.g., number of branches, length, width, direction, etc.) can be designed based on the specific application.

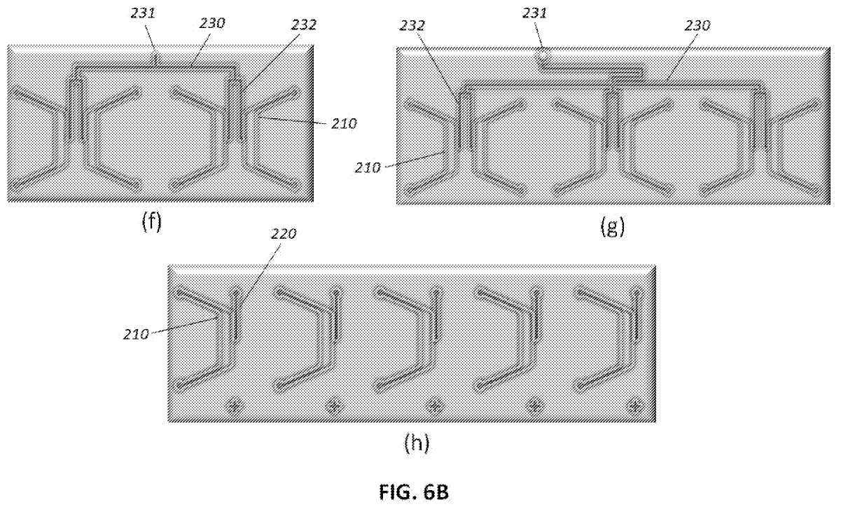

FIGS. 6A-6B illustrate various designs of the first layer in the microfluidic device. The first layer functions as cell culture layer and may include one or more cell culture chambers. The cell culture layer in view (a) has a single cell culture chamber 210. The cell culture chamber 210 includes a cell cavity 211, an inlet 212, an outlet 213, an inlet channel 214 connecting the cell cavity 211 and the inlet 212, and an outlet channel 215 connecting the cell cavity 211 and the outlet 213. Cell culture media or agents can flow through the cell culture chamber 211 via the inlet 212 and the outlet 213. The cell cavity 211 may have an elongated shape as shown in view (a). Each of the inlet and outlet channels 214, 215 may form an angle with the cell cavity 211. The angle can be in the range of 90 to 180 degrees, e.g., 100 to 150 degrees, 110 to 140 degrees, or 115 to 130 degrees. The obtuse angle avoids an abrupt change in direction from the inlet 212 to the outlet 213.

Design of the cell culture chamber may vary according to the specific application. In certain embodiments, the shape of the cell cavity matches the shape of the air cavity. In alternative embodiments, one cell cavity can be influenced by a group of air cavities. Each air cavity in the group covers only a portion of the cell cavity when viewed from the above.

Compared with view (a), the cell culture layer in view (b) further includes a microchannel 220. The microchannel 220 extends from an inlet 221 and adjoins the cell culture chamber 210. In certain embodiments, the microchannel 220 can be oriented in parallel with the cell cavity 211. The microchannel 220 can adjoin the cell cavity 211 over a portion of its length, e.g., over ⅕ of its length, over ⅓ of its length, or over half of its length.

The microchannel 220 can be an extracellular matrix (ECM) channel. The ECM channel is used to contain one or a combination of ECM hydrogels, such as hydrogels of collagen type I, collagen type IV, Matrigel, fibrinogen, hyaluronic acid, synthetic hydrogels such as gelatin-based (GelMA) or poly(ethylene glycol) (PEG)-based. Such hydrogels are important for long-term cell and tissue maintenance.

In one embodiment, the ECM channel opens to the cell culture chamber through a capillary burst valve (CBV) which ensures that the ECM hydrogels loaded into the ECM channel does not overflow into the cell culture chamber. The capillary burst valve can be integrated into the microfluidic device design to allow the incorporation of multiple parallel channels that are interconnected without the need for external tubings or fittings. However, it should be noted that the capillary burst valve is not an essential element to the invention and can be omitted or removed. Other types of flow control devices can also be used to regulate the flow of ECM hydrogels into the cell culture chamber.

Each of the cell culture layer shown in views (c), (d) and (e) includes multiple cell culture chambers 210 and an ECM channel 220. In view (c), two cell culture chambers 210 are arranged such that the two cell cavities 211 face each other. The ECM channel 220 is sandwiched between the two cell cavities 211. ECM hydrogels can flow through the ECM channel 220 into the two cell cavities 211. A capillary burst valve may be disposed between each side of the ECM channel and a respective cell culture chamber. In view (d), the ECM channel 220 bifurcates. Each branch 222 of the ECM channel 220 adjoins one cell cavity 211. A capillary burst valve may be disposed between each branch and a respective cell culture chamber. The cell culture layer in view (e) includes three cell culture chambers and an ECM channel 220 having three branches 222. Each branch opens to a respective cell culture chamber. A capillary burst valve may be disposed at the end portion of each branch to control the flow of ECM hydrogels.

Each of the cell culture layer shown in views (f), (g) and (h) includes an array of cell culture chambers. The design in view (f) has four cell culture chambers and can be considered as a combination of two units shown in view (d). The ECM channel is configured as a channel network 230 having a single inlet 231 and four branches 232. Each branch 232 adjoins one cell culture chamber 210. Likewise, the cell culture layer in view (f) is a combination of three units shown in view (d). It should be appreciated that the shape, number and arrangement of cell culture chamber in the cell culture layer can be changed to fit different applications. The ECM channel network 230 may have a complicated design if a large number of cell culture chambers are used. Alternatively, the cell culture layer may include multiple ECM channels 220 separated from each other, such as the embodiment shown in view (h). Separate ECM channels 220 allow distinct ECM hydrogels to enter different cell culture chambers 210.

The actuator layer and cell culture layer can be made of a polymer such as polydimethylsiloxane (PDMS), polystyrol (PS), polycarbonate (PC), and/or polysiloxane. The materials of the actuator layer and cell culture layer can have any degree of transparency, reflectivity, or other optical characteristics. For instance, the materials can be transparent to enable optical analysis. Additionally or alternatively, a portion of the layers can be opaque, translucent, or any suitable opacity.

Design of the cell culture layer can be changed for different applications. Moreover, depending on the configuration of the air chambers in the actuator layer, mechanical actuation applied to each cell culture chamber can be controlled separately. The microfluidic devices according to the present disclosure can be used to build high-throughput cell culture systems capable of maintaining cells or tissues over prolonged periods of time, and also maintaining the desired mechanical actuation and perfusion culture. Four example applications of the microfluidic device are described below.

### EXAMPLE 1

### Microfluidic Device With Parallelized Cell Culture Chambers

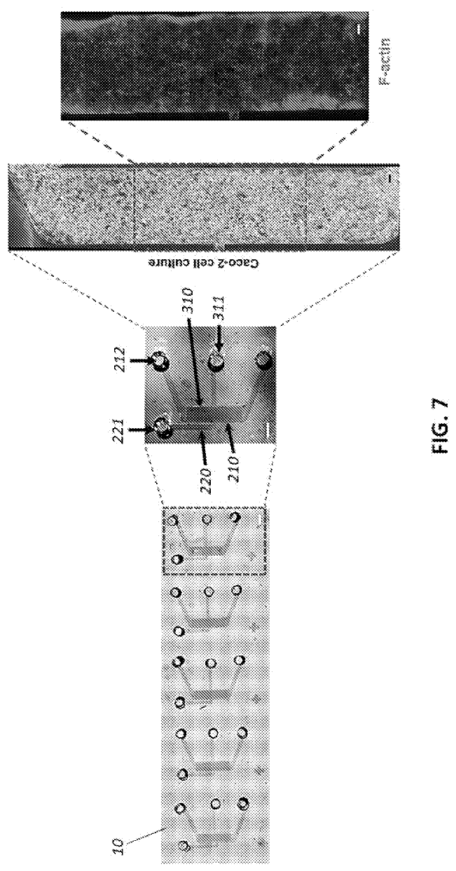

The microfluidic device 10 illustrated in FIG. 1 was used to develop a biomimetic gut epithelium using the Caco-2 cell line, which is widely used as a model of intestinal epithelial barrier. FIG. 7 shows the microscopic images of the microfluidic device and the cell culture chamber. A hydrogel was introduced into the ECM channel 220 from the ECM inlet 221. A protein coating solution comprising one or a combination of proteins such as Type I collagen, fibronectin, gelatin or matrigel was introduced to the cell culture chamber 210 via the ECM channel 220 to facilitate the attachment and culture of cells. Then Caco-2 cells were introduced from the cell chamber inlet 212 and were allowed to adhere onto the glass substrate. The culture was maintained for 6-8 days under constant perfusion and under the influence of cyclic compression and relaxation. As described above, the cyclic compression and relaxation of each cell culture chamber 210 can be achieved by independent control of the air introduced into and removed from the air chamber 310 via the air inlet 311. Immunostaining was then performed and high-resolution image of the Caco-2 cells were obtained by fluorescence microscopy.

While the test was performed using a microfluidic device having five cell culture chambers arranged in parallel, it should be appreciated that the microfluidic device according to the present disclosure can be expanded to incorporate a greater number of cell culture chambers. The arrangement of the cell culture chambers can be modified to include multiple rows and columns.

### EXAMPLE 2

### Microfluidic Device With Interconnected Cell Culture Chambers

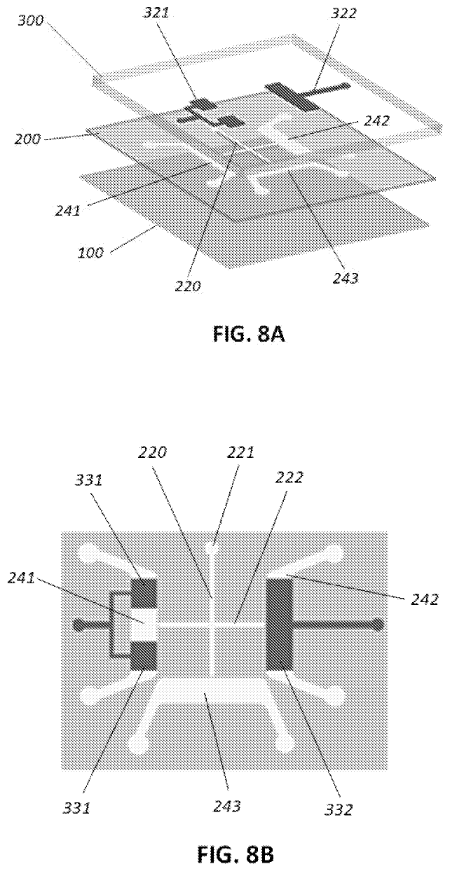

FIGS. 8A-8B show a microfluidic device according to an alternative embodiment of the present disclosure. The microfluidic device has a glass layer 100, a cell culture layer 200 disposed on the glass layer 100 and an actuator layer 300 disposed on the cell culture layer 200. The cell culture layer may adopt the design depicted in view (e) in FIG. 6A. The ECM channel 220 formed in the cell culture layer 200 includes an ECM inlet 221 and three branches 222 in communication with the three cell culture chambers 241, 242, 243 respectively. In this example, the ECM channel is configured as a means for interconnecting multiple cell culture chambers. Since ECM is porous, it allows diffusion of liquid therethrough while preventing any convective flow.

While the three cell culture chambers share the same design, their associated air chamber patterns can be different. As best seen in FIG. 8B, the cell culture chamber 241 on the left hand side is under influence of an air chamber 321 consisting of two smaller air cavities 331, with each cavity 331 covering only a portion of the cell culture chamber 241. The air chamber design is similar to that depicted in view (f) of FIG. 5B. The cell culture chamber 242 on the right hand side is under influence of an air chamber 322 having one larger air cavity 332. No air chamber is provided for the cell culture chamber 243 at the bottom. As a result, unique mechanical stimuli can be generated in different cell culture chambers.

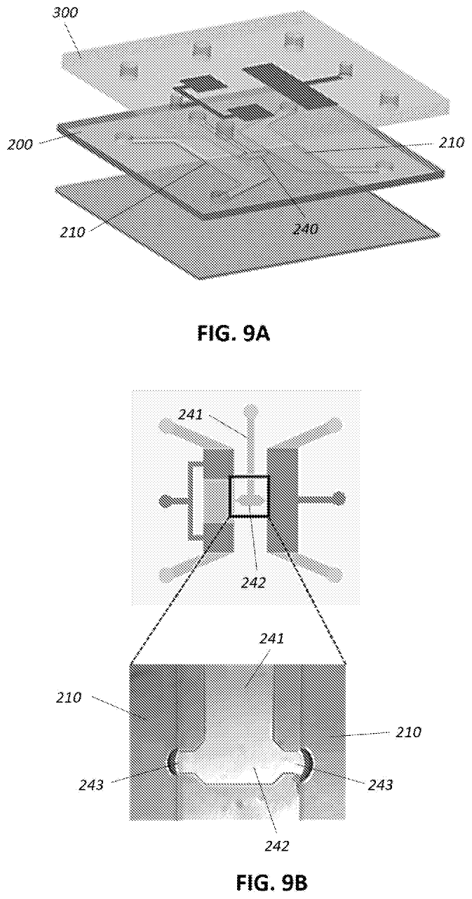

FIGS. 9A-9B show a microfluidic device according to a further embodiment of the present disclosure. The actuator layer 300 resembles that used in FIGS. 8A-8B. The cell culture layer 200 includes two cell culture chambers 210 facing each other and interconnected by an ECM channel 240. The ECM channel 240 includes a main section 241 and an end section 242 extending between the two cell culture chambers 210 in a direction generally perpendicular to the main section 241. Opposite ends 243 of the end section 242 open to the two cell culture chambers 210.

Small-molecule diffusion can be observed and studied by using fluorescence microscopy. FIG. 9C illustrates microscopic images showing the diffusion of 4.4 kDa FITC-dextran molecule to study its diffusion profile through the ECM. FIG. 9D depicts a graph showing the fluorescence intensity over the distance a-a′ (indicated in FIG. 9C) along the end section of the ECM channel.

### EXAMPLE 3

### Co-culture Different Cell Types Using the Microfluidic Device

Apart from providing unique mechanical actuation in the cell culture chambers, the microfluidic device according to the present disclosure can also be used to co-culture different cell types pertaining to different human organs. In this example, the microfluidic device was used to culture an endothelial tube using human microvascular endothelial cells in one chamber and Caco-2 cells in the adjacent chamber. Different mechanical forces can be applied to the cells. The culture was maintained for 7 days. In addition to adherent cells, suspension cells such as immune cells can also be maintained within the cell culture chamber. Accordingly, this example shows the ability of the microfluidic device to create a tri-culture system involving three different cell lines, which makes it possible to generate a multi-organ on a chip model.

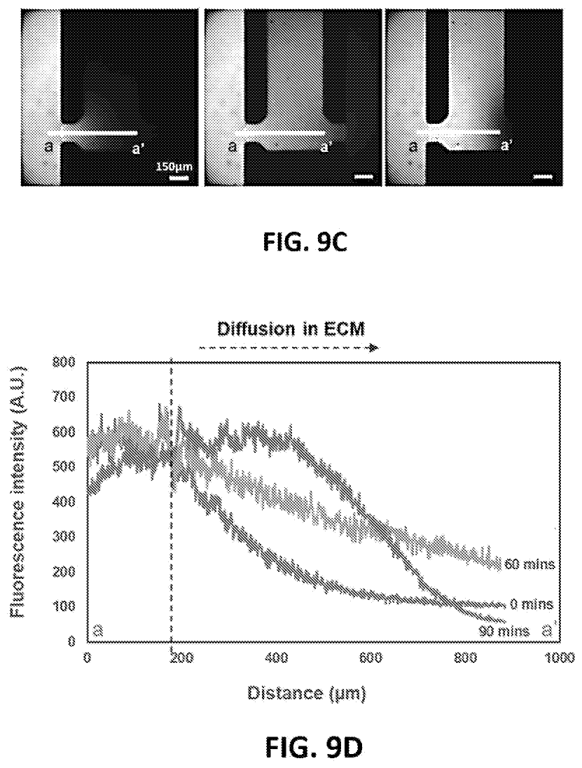

FIG. 10 illustrates microscopic images showing the tri-culture system created by the microfluidic device. Human microvascular endothelial cells and human colon adenocarcinoma cells resembling gut epithelial cells were cultured on either side of the ECM and maintained at unique mechanical actuation. The arrows in the image of cell chamber 1 indicate the suspension human monocyte cell line (THP-1) that was cultured within the endothelial cell tube. Insets in FIG. 10 show the growth of the cells in the respective cell chambers under the mechanical actuation.

### EXAMPLE 4

### Co-culture of Micro-organisms Using the Microfluidic Device

A further application of the microfluidic device according to the present disclosure includes the co-culture of micro-organisms with the mammalian cells. The fluid flow, shear stress, and mechanical actuation mimicking dynamic organ system enable comparatively long-term co-culture (>48 hours) of mammalian cells with single or multiple types of microorganism, which is challenging in conventional cell culture systems due to the rapid growth rates of the microbe as compared to the mammalian cells.

In this example, the microfluidic device was used to demonstrate the co-culture of Caco-2 cells, mimicking gut epithelial cells and various gut microbes such as Escherichia coli and Lactobacillus rhamnosus. The Caco-2 cell was cultured for 7 days under different mechanical forces and intestinal microbes were introduced on day 6 and co-cultured for up to 48 hours.

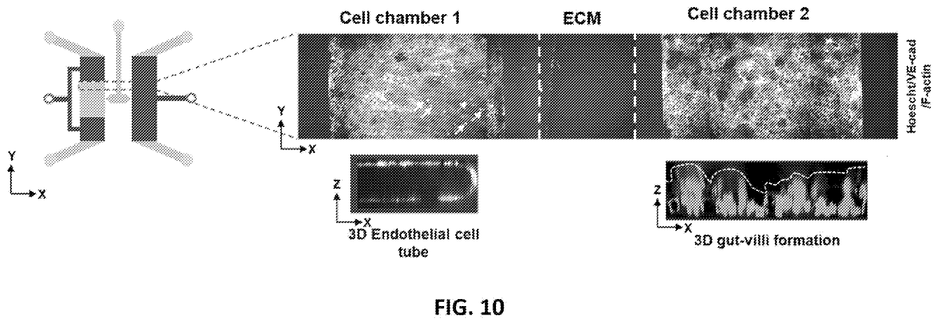

FIG. 11 illustrates microscopic images showing bacterial colonization on the microfluidic device. The images show: (A) Colonization of GFP-E. coli on mucus layer of the Caco-2 cells; (B) colonization of GFP-E. coli on the recapitulated microvilli; and (C) colonization of Lactobacillus rhamosus on the recapitulated microvilli. This example shows the possibility of recreating a host-microbe model system using the microfluidic device.

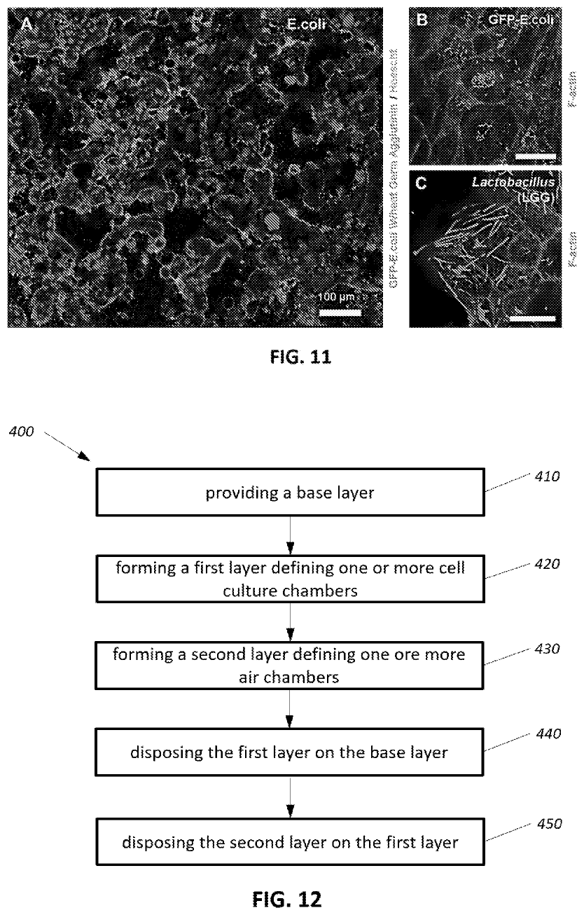

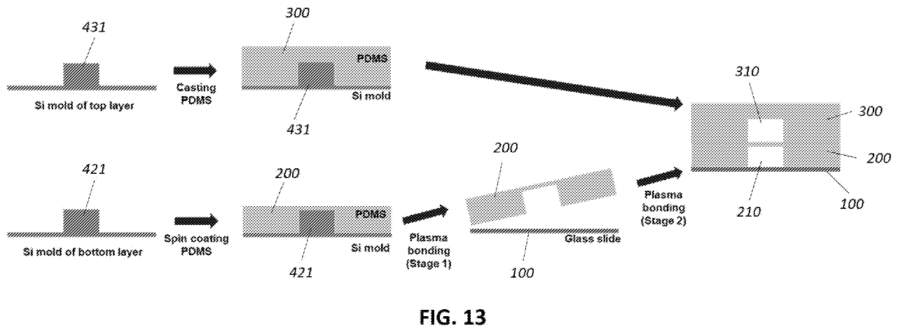

FIG. 12 depicts an example method 400 of fabricating a microfluidic device in accordance with one embodiment of the present disclosure. FIG. 13 shows a schematic view of the fabrication process. In one or more embodiments, one or more of the elements shown in FIGS. 12 and 13 may be omitted, repeated, and/or performed in a different order. Accordingly, embodiments of the fabrication method should not be considered limited to the specific arrangements of elements shown in the figures.

In Block 410, a base layer is provided. The base layer 100 (FIG. 13) can be made of a transparent material which enables optical analysis of the cells or tissues being cultured in the microfluidic device. In certain embodiments, the base layer can be a coverslip, preferably a microscopy-grade glass coverslip, making the microfluidic device compatible with super-resolution microscopy.

In Block 420, a first layer is formed. The first layer 200 (FIG. 13) functions as the cell culture layer. The first layer 200 defines one or more cell culture chambers 210 (FIG. 13). Each of the one or more cell culture chambers 210 is configured to contain cells, tissues or micro-organisms. In certain embodiments, as illustrated in FIG. 13, the first layer 200 (bottom layer) can be formed by spin-coating a polymer over a first mold 421. The first mold 421 can be a silicon mold fabricated using SU-8 based photolithography.

In certain embodiments, forming the first layer can include forming a plurality of extracellular matrix (ECM) channels in the first layer. Each ECM channel adjoins a cell culture chamber to allow one or a combination of ECM hydrogels to be introduced into the cell culture chamber.

In Block 430, a second layer is formed. The second layer 300 (FIG. 13) functions as the actuator layer. The second layer 300 defines one or more air chambers 310. In certain embodiments, as illustrated in FIG. 13, the second layer 300 (top layer) can be formed by casting a polymer over a second mold 431. Likewise, the second mold 431 can be a silicon mold fabricated using SU-8 based photolithography.

In certain embodiments, the first and second layers 200, 300 are both made of polydimethylsiloxane (PDMS). To form the first layer 200, PDMS, preferably in the ratio between 10:1 and 15:1 (w/w), is spin-coated over a silicon mold to generate PDMS having a thickness smaller than 1 mm, e.g. smaller than 500 μm, for housing the cell culture chambers. To form the second layer 300, a thicker layer of PDMS, preferably having a thickness in the range of 2 mm to 3 mm, is cast over another silicon mold to create the desired configuration of air chambers.

In certain embodiments, access ports (e.g. inlets and outlets) are formed on the first and second layers. The ports on the first layer can be used to guide liquid or gas into and from the cell culture chamber. The ports on the second layer can be used to introduce and remove air into and from the air chamber.

In Block 440, the first layer is disposed on the base layer. In certain embodiments, as illustrated in FIG. 13, a first stage plasma bonding process can be performed to attach the first layer 200 (e.g. a thinner PDMS layer) to the base layer 100 (e.g. a glass coverslip).

Finally, in Block 450, the second layer is disposed on the first layer. Proper alignment between the air chambers in the second layer and the cell culture chambers in the first layer may be required to ensure that each air chamber is adjacent a respective cell culture chamber. In certain embodiments, as illustrated in FIG. 13, a second stage plasma bonding process can be performed to attach the second layer 300 (e.g. a thicker PDMS layer) to the first layer 200 (e.g. a thinner PDMS layer).

The microfluidic device fabricated using the method described above has a multi-layer configuration, including a glass coverslip at the bottom, a PDMS cell culture layer in the middle, and a PDMS actuator layer at the top. By tuning the design of the actuator layer, a unique mechanical actuation pattern can be applied to individual cell culture chamber in the cell culture layer. The microfluidic device according to the present disclosure represents a big improvement over the commercially available platforms where the same mechanical actuation has to be applied to all the cell culture chambers.

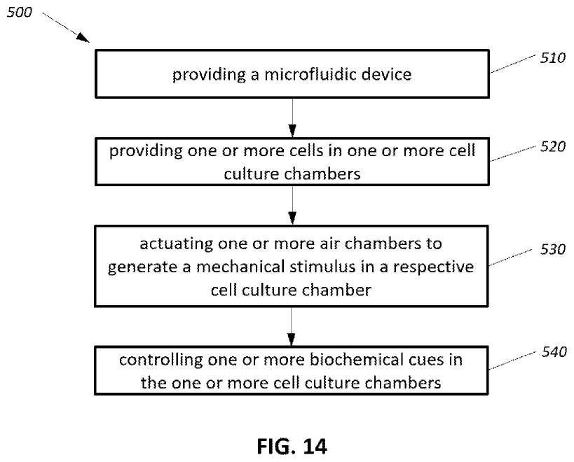

FIG. 14 depicts an example cell culture method 500 in accordance with one embodiment of the present disclosure. For example, the method in FIG. 14 may be practiced using the microfluidic device described with reference to FIGS. 1-10 above.

Initially in Block 510, a microfluidic device is provided. The microfluidic device can be a multi-layer organ chip including a base layer, a cell culture layer supported on the base layer, and an actuator layer disposed on the first layer. The microfluidic device can be fabricated using the method described with reference to FIGS. 12-13.

The cell culture layer may define one or more cell culture chambers. The cell culture layer may have a layout according to any embodiment illustrated in FIGS. 6A-6B or a combination of the embodiments. The actuator layer may define one or more air chambers. The actuator layer may have a layout according to any embodiment illustrated in FIGS. 5A-5B or a combination of the embodiments.

In Block 520, one or more cells are provided in the one or more cell culture chambers. In certain embodiments, a first cell type can be cultured in a first cell culture chamber. A second cell type, which is different from the first cell type, can be cultured in a second cell culture chamber. The first and second cell culture chambers may share the same design or may have distinct patterns. As such, different types of cells can be cultured simultaneously using a single microfluidic device.

In certain embodiments, one or more types of microorganisms can be provided together with a cell in a cell culture chamber. Example 4 described above shows that the microfluidic device according to the present disclosure allows for co-culture of micro-organisms with cells.

In certain embodiments, fluid flow in the one or more cell cultures can be controlled via respective inlets and outlets of the one or more cell culture chambers.

In Block 530, the one or more air chambers in the actuator layer are actuated. Actuation causes the air chambers to expand or contract, thereby generating a mechanical stimulus in a respective cell culture chamber. In certain embodiments, the one or more air chamber chambers can be actuated independently of one another, for example by using a programmable controller. As a result, biomechanical cues for cell growth in distinct cell culture chambers can be controlled independently. The biomechanical cues may include fluidic flow, shear stress, pressure gradient, and/or mechanical compression and expansion.

In certain embodiments, the mechanical stimulus can be generated by actuating the one or more air chambers and controlling fluid flow through the one or more cell culture chambers, simultaneously.

In Block 540, one or more biochemical cues in the one or more cell culture chambers are controlled. The biochemical cues may include proteins, peptides, oligonucleotides, chemicals, nanoparticles or drugs. In certain embodiments, controlling the biochemical cues may include tuning the oxygen concentration in the one or more cell culture chambers using an oxygen control module. As such, the method significantly shortens the time required to conduct a comparative study of cell growth under different biochemical cues.

The present disclosure provides a multi-layered, multi-chambered platform with or without integrated mechanical actuators. The integrated mechanical actuators can include multiple air chambers which enable distinct mechanical forces such as fluidic flow, shear stress, pressure gradient, mechanical compression and expansion to be maintained in distinct cell culture chambers simultaneously. The platform may define an array of cell culture chambers to achieve high throughput drug or compound screening. The multi-layer structure allows user to easily and comfortably modify the dimensions or designs of the cell culture chambers as well as the mechanical actuators according to the requirements of different applications.

Further, a capillary burst valve can be integrated into the platform to enable the incorporation of multiple parallel cell culture chambers that are interconnected without the need for external tubings or fittings. Additionally, the use of thin glass coverslip as the base layer makes the platform suitable for super-resolution microscopy and compatible with high magnification objective lenses with low working distances.

Various examples described above show that the platform can be utilized, for example, to create an immune competent, vascularized model of human intestine by inclusion of relevant cell types in addition to the culture of wide variety of microbe. This helps to develop a microphysiological system of human intestine with relevant human intestinal cells (primary cells or cell lines), microbe, endothelial cells and immune cells. The microphysiological system can be used in the study of host-microbe interaction, microbe induced disease modeling and drug development.

The platform also allows for long term pumpless or pump-based perfusion for culture of mammalian cell, micro-organisms or both. Accordingly, the platform provides users with the flexibility to tune the application and recapitulate multiple microphysiological systems such as liver, gut, blood vessel, kidney and heart.

The description uses embodiments to disclose the invention and enable any person skilled in the art to practice the invention, including making and using any devices or systems and performing any incorporated methods. The patentable scope of the invention is defined by the claims, and may include other embodiments that occur to those skilled in the art. Such other embodiments are intended to be within the scope of the claims if they include elements that do not differ from the literal language of the claims, or if they include equivalent elements with insubstantial differences from the literal language of the claims.

1. A microfluidic device, comprising:
a base layer;
a first layer disposed on the base layer, the first layer defining one or more cell culture chambers, each of the one or more cell culture chambers configured to contain one or more cell cultures; and
a second layer disposed on the first layer, the second layer defining one or more air chambers,
wherein each of the one or more air chambers is adjacent a respective cell culture chamber.

2. The device according to claim 1, wherein the base layer comprises a transparent glass layer.

3. The device according to claim 1, wherein the first layer defines a plurality of cell culture chambers, and wherein the plurality of cell culture chambers are disposed in a lateral configuration.

4. The device according to claim 3, wherein the second layer defines a corresponding plurality of air chambers, and wherein the device further comprises a programmable pneumatic controller for operating the plurality of air chambers, the programmable pneumatic controller being configured to actuate the plurality of air chamber chambers independently of one another.

5. The device according to claim 4, wherein each of the plurality of air chambers is configured to generate a biomechanical cue in the respective cell culture chamber, the biomechanical cue comprising at least one of a fluidic flow, a shear stress, a pressure gradient, or mechanical compression and expansion.

6. The device according to claim wherein at least selected ones of the plurality of cell culture chambers are connected to one another.

7. The device according to claim 6, wherein the first layer further defines a plurality of extracellular matrix (ECM) channels, each of the ECM channels adjoining a respective one of the plurality of cell culture chambers.

8. The device according to claim 7, further comprising a capillary burst valve disposed between each of the plurality of ECM channels and a respective one of the plurality of cell culture members.

9. A cell culturing method, comprising:
providing a microfluidic device as defined in claim 1;
providing one or more cell cultures in the one or more cell culture chambers; and
actuating the one or more air chambers such that each of the one or more air chambers generates a mechanical stimulus on a respective cell culture chamber.

10. The method according to claim 9, wherein providing one or more cell cultures in the one or more cell culture chambers comprises providing a cell culture of a first cell type in a first cell culture chamber and providing a cell culture of a second cell type different from the first cell type in a second cell culture chamber.

11. The method according to claim 9, wherein providing one or more cell cultures in the one or more cell culture chambers further comprises providing one or more types of microorganisms together with a cell culture in a cell culture chamber.

12. The method according to claim 9, wherein providing one or more cell cultures in the one or more cell culture chambers further comprises controlling fluid flow in the one or more cell cultures via respective inlets and outlets of the one or more cell culture chambers.

13. The method according to claim 9, wherein actuating the one or more air chambers comprises actuating the one or more air chamber chambers independently of one another.

14. The method according to claim 12, comprising generating a mechanical stimulus in one or more cell culture chambers with one or more cell culture by actuating the one or more air chambers and controlling fluid flow through the one or more cell culture chambers, simultaneously.

15. The method according to claim 9, further comprising controlling one or more biochemical cues selected from a group consisting of proteins, peptides, oligonucleotides, chemicals, nanoparticles or drugs, for the one or more cell cultures.

16. The method according to claim 15, wherein controlling one or more biochemical cues comprises tuning the oxygen concentration in the one or more cell culture chambers using an oxygen control module.

17. A method of fabricating a microfluidic device, the method comprising:
providing a base layer;
forming a first layer such that the first layer defines one or more cell culture chambers, each of the one or more cell culture chambers configured to contain one or more cell cultures;
forming a second layer such that the second layer defines one or more air chambers, disposing the first layer on the base layer; and
disposing the second layer on the first layer such that each of the one or more air chambers is adjacent a respective cell culture chamber.

18. The method according to claim 17, wherein forming the first layer comprises spin-coating polydimethylsiloxane (PDMS) over a first mold, and wherein forming the second layer comprises casting PDMS over a second mold.

19. The method according to claim 17, wherein disposing the first layer on the base layer comprises a first plasma bonding step, and wherein disposing the second layer on the first layer comprises a second plasma bonding step.

20. The method according to claim 17, wherein forming the first layer further comprises forming a plurality of extracellular matrix (ECM) channels each adjoining a respective one of the plurality of cell culture chambers.
